(19) United States
(12) Patent Application Publication
(10) Pub. No.: US 2025/0152954 A1
(43) Pub. Date: May 15, 2025

(54) AUTOMATED EXTERNAL DEFIBRILLATOR

(71) Applicant: NIHON KOHDEN CORPORATION, Tokyo (JP)

(72) Inventors: Ryosuke KUNO, Tokorozawa-shi, Saitama (JP); Yuji IGAWA, Tokorozawa-shi, Saitama (JP)

(73) Assignee: NIHON KOHDEN CORPORATION, Tokyo (JP)

(21) Appl. No.: 18/726,966

(22) PCT Filed: Jan. 6, 2023
(86) PCT No.: PCT/JP2023/000148

§ 371 Date: Jul. 5, 2024

(30) Foreign Application Priority Data
Jan. 7, 2022 (JP) 2022-001892

(51) Int. Cl. A61N 1/39; A61N 1/362

(52) U.S. Cl. CPC A61N 1/3993; A61N 1/3625; A61N 1/3904

(57) ABSTRACT

An automated external defibrillator configured to deliver an electric shock for defibrillation to a heart of a subject. The automated external defibrillator includes: a sound generator configured to output: a timing sound expressing, by sound, a timing until the electric shock is delivered; and a warning sound expressing a state in which an internal capacitor of the automated external defibrillator has been charged with high voltage; and a sound controller configured to control the sound generator. The sound controller changes the warning sound in accordance with presence or absence of output of the timing sound.

### TECHNICAL FIELD

The present disclosure relates to an automated external defibrillator.

### BACKGROUND ART

Currently, automated external defibrillators (hereinafter also referred to as an AED) have been rapidly widespread. The AED delivers a strong electric shock for defibrillation to the heart of an injured or sick person who has suffered a sudden cardiac arrest due to ventricular fibrillation, so as to restore a cardiac function of the injured or sick person. The AED discharges high voltage when delivering the electric shock to the injured or sick person. Therefore, there has been known a mechanism for preventing a rescuer from receiving the electric shock. For example, Japanese Patent Publication No. 2014-521476A (Patent Document 1) discloses an AED provided with a safety interlock to prevent electric discharge from being performed unless a rescuer releases his or her hands from an injured or sick person.

When the AED disclosed in the Patent Document 1 is used, start of the electric discharge may be delayed because the rescuer does not notice that the electric discharge will not be performed unless the rescuer releases his or her hands from the injured or sick person. In particular, in a case of an automated external defibrillator that that is not provided with any shock button (hereinafter also referred to as auto-shock AED), the rescuer may not recognize that the electric discharge will be automatically performed, or the rescuer may not recognize a timing when the automatic electric discharge is performed only with a simple countdown notification presented visually on a screen or audibly.

### SUMMARY

An object of the present disclosure is to provide an AED that clearly notifies a rescuer of a timing for electric discharge so that the rescuer can recognize that the electric discharge will be performed.

According to an aspect of the present disclosure, there is provided an automated external defibrillator configured to deliver an electric shock for defibrillation to the heart of a subject, the automated external defibrillator including:
a sound generator that outputs a timing sound expressing, by sound, a timing until the electric shock is delivered, and a warning sound expressing a state in which an internal capacitor of the automated external defibrillator has been charged with high voltage; and
a sound controller that controls the sound generator, wherein
the sound controller changes the warning sound in accordance with presence or absence of output of the timing sound.

### BRIEF DESCRIPTION OF DRAWINGS

FIG. 1 An external view of an AED according to one embodiment of the present disclosure.

FIG. 2 A block diagram illustrating a functional configuration of the AED according to the embodiment of the present disclosure.

FIG. 3 A chart illustrating a flow of a rescue process of the AED according to the embodiment of the present disclosure.

FIG. 4 An illustrative view illustrating a display of the AED according to the embodiment of the present disclosure.

FIG. 5 A chart illustrating a flow of a display process of the AED according to a first embodiment of the present disclosure.

FIG. 6 A view illustrating a transition flow of the display of the AED according to the first embodiment of the present disclosure.

FIG. 7 A chart illustrating a flow of a sound output process of the AED according to a second embodiment of the present disclosure.

FIG. 8 A chart illustrating a flow of a sound output process of the AED according to a third embodiment of the present disclosure.

### DESCRIPTION OF EMBODIMENT

An embodiment of the present disclosure will be described below with reference to the drawings. It should be noted that for convenience of explanation, description about members having the same reference numerals as those already described in the description of the embodiment will be omitted. In addition, dimensions of each member shown in the drawings may be different from actual dimensions of the member for convenience of explanation.

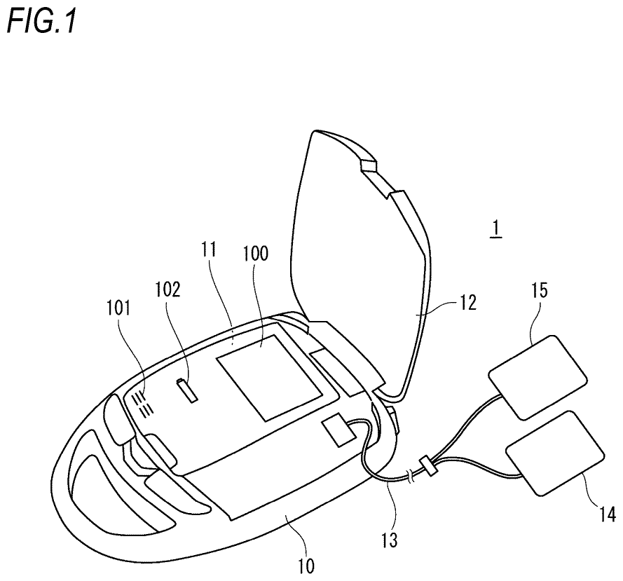

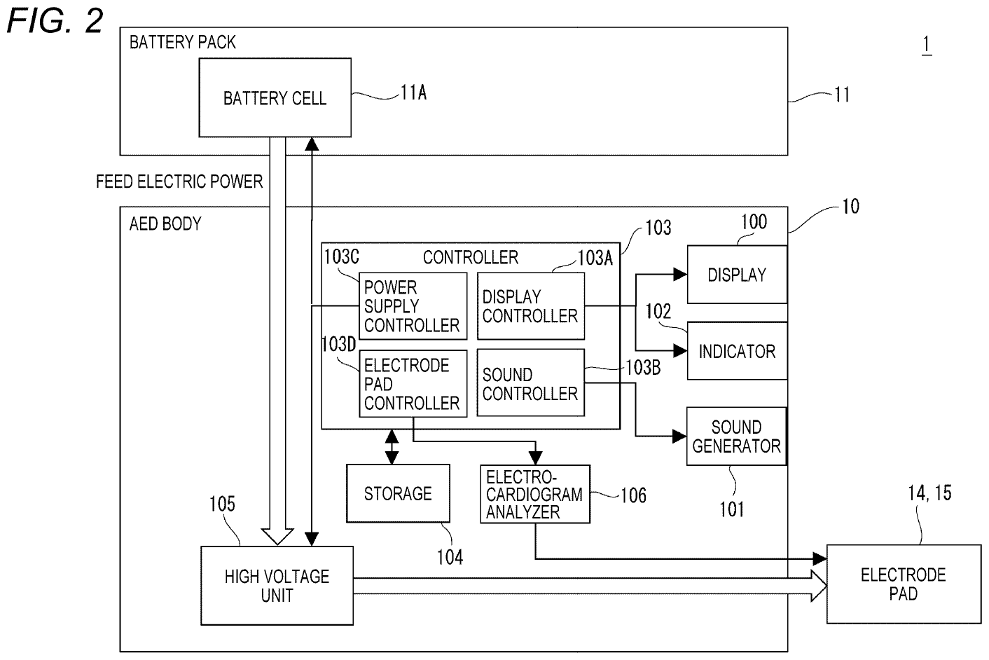

FIG. 1 is an external view of an AED 1 according to one embodiment of the present disclosure. The AED 1 is provided with an AED body 10 (housing body), and a lid part 12 covering the AED body 10. The AED 1 is configured so that the AED body 10 and the lid part 12 can be connected with a battery pack 11, a cable 13, and electrode pads 14 and 15. The battery pack 11 including a battery cell 11A (see FIG. 2) feeds electric power to the AED body 10 so as to activate the AED body 10 and the electrode pads 14 and 15. In addition, the battery pack 11 is connected to a back side of the AED body 10 in a removable structure. The lid part 12 has a structure to cover the AED body 10. When the lid part 12 is opened, a main power supply of the AED 1 is turned ON. When the lid part 12 is closed, the main power supply of the AED 1 is turned OFF. It should be noted that the AED 1 is not limited to the configuration in which the power supply is turned ON/OFF by opening/closing the lid part 12. For example, a power button may be alternatively provided in the housing body 10. The cable 13 electrically connects the AED body 10 to the electrode pads 14 and 15. The electrode pads 14 and 15 are attached to an injured or sick person (subject) to make it possible to perform electrocardiogram analysis and treatment such as an electric shock (hereinafter also referred to as “discharging”) on the injured or sick person.

The AED body 10 is provided with a display 100, a sound generator 101, and an indicator 102 as user interfaces. The display 100 is a display capable of showing an illustration, a character or text, etc., and visually shows, to the rescuer, how to attach the electrode pads 14 and 15, how to deliver an electric shock, etc. The sound generator 101 is a speaker that audibly notifies the rescuer of how to attach electrode pads 14 and 15, how to deliver the electric shock, a warning sound expressing a state in which the AED body 10 has been internally charged with high voltage, etc. The indicator 102 includes a lamp or the like that can be lit up or blinked to visually show, to the rescuer, whether the AED 1 is ready for use or not, etc.

FIG. 2 is a block diagram illustrating a functional configuration of the AED 1. The battery pack 11 has the battery cell 11A. The AED body 10 has the display 100, the sound generator 101, the indicator 102, a controller 103, a storage 104, a high voltage unit 105, and an electrocardiogram analysis part 106. The controller 103 controls various operations of the AED 1. The controller 103 has a display controller 103A, a sound controller 103B, a power supply controller 103C, and an electrode pad controller 103D. In response to the lid part 12 opened to turn ON the main power supply of the AED 1, the controller 103 reads a program etc. stored in the storage 104, and executes the program etc. to control the various operations of the AED 1.

The display controller 103A reads image data stored in the storage 104 in advance. The image data is associated with a guidance to the rescuer, such as how to attach the electrode pads 14 and 15, how to deliver the electric shock, as well as time information regarding a time length until discharging is performed. The display controller 103A transmits the read image data to the display 100. The display 100 shows the received image data on a screen.

In addition, the display controller 103A transmits a control signal to the indicator 102 to notify the rescuer of whether the AED 1 is ready for use or not. The lamp etc. of indicator 102 is lit up, lit out, or blinked based on the received control signal.

Sound data about guidance to the rescuer, such as how to attach the electrode pads 14 and 15, how to deliver the electric shock, the warning sound indicating the state in which the AED body 10 has been internally charged with high voltage, and a timing sound until the discharging is performed have been stored in the storage 104 in advance. The sound controller 103B reads the sound data, and transmits the read sound data to the sound generator 101. The sound generator 101 outputs sound based on the received sound data.

The power supply controller 103C controls the battery cell 11A and an internal capacitor of the high voltage unit 105 so as to charge the internal capacitor by the battery cell 11A and perform electric discharge from the electrode pads 14 and 15.

The electrode pad controller 103D determines whether the electrode pads 14 and 15 have been attached to the injured or sick person or not, and controls the electrode pads 14 and 15 and the electrocardiogram analysis part 106 so as to cause the electrocardiogram analysis part 106 to perform electrocardiogram analysis.

The storage 104 stores the program etc. for controlling the various operations of the AED 1. The electrocardiogram analysis part 106 performs the electrocardiogram analysis on the injured or sick person through the electrode pads 14 and 15.

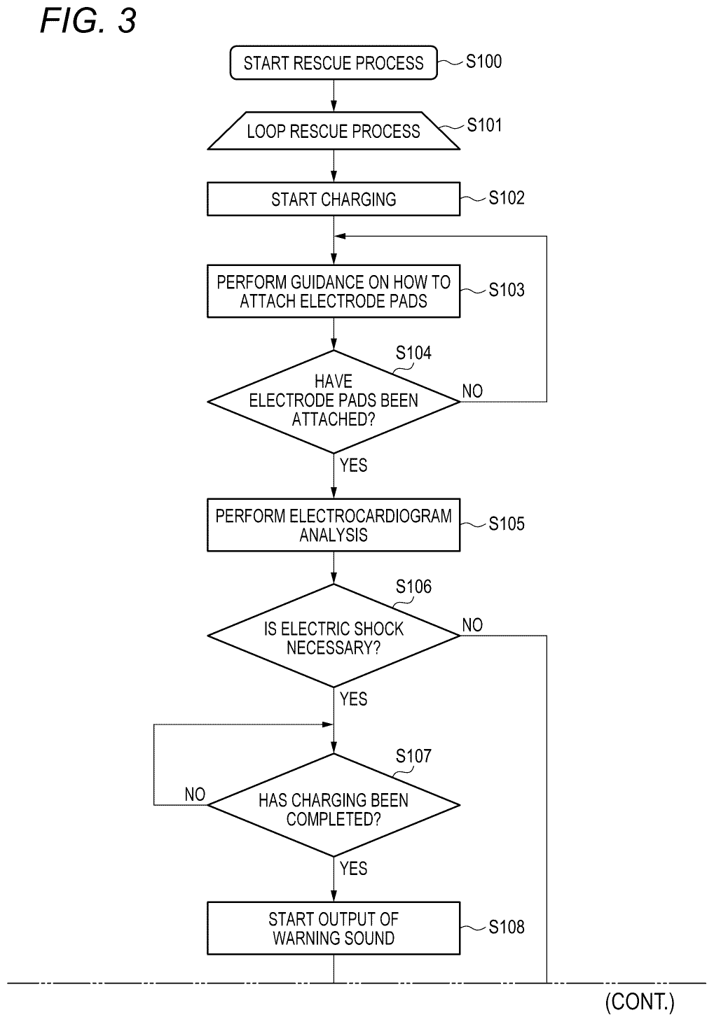

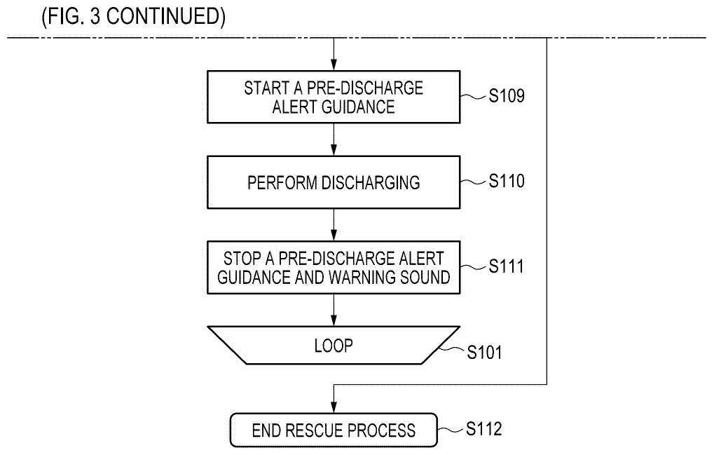

FIG. 3 illustrates a flow of a rescue process of the AED according to the embodiment of the present disclosure. In response to the lid part 12 opened to turn ON the main power supply of the AED 1, the controller 103 reads the program etc. stored in the storage 104, to thereby start the rescue process (S100).

The power supply controller 103C starts charging the internal capacitor of the high voltage unit 105 with the battery cell 11A (S102).

Next, the display controller 103A and the sound controller 103B perform a guidance to the rescuer on how to attach the electrode pads 14 and 15 to the injured or sick person (S103). Specifically, the display controller 103A transmits, to the display 100, image data about how to attach the electrode pads 14 and 15 to the injured or sick person, and the display 100 shows the image data to the rescuer. In addition, the sound controller 103B transmits, to the sound generator 101, sound data indicative of how to attach the electrode pads 14 and 15 to the injured or sick person, and the sound output section 101 outputs sound to the rescuer based on the received sound data.

Next, the electrode pad controller 103D measures impedance between the electrode pads 14 and 15, and determines whether the electrode pads 14 and 15 have been attached to the injured or sick person or not, based on the measured impedance (S104). When the electrode pad controller 103D determines that the electrode pads 14 and 15 have not been attached to the injured or sick person, the display controller 103A and the sound controller 103B perform the guidance to the rescuer again on how to attach the electrode pads 14 and 15 to the injured or sick person (S103). When determining that the electrode pads 14 and 15 have been attached to the injured or sick person, the electrode pad controller 103D instructs the electrocardiogram analysis part 106 to perform electrocardiogram analysis. The electrocardiogram analysis part 106 performs the electrocardiogram analysis based on the instruction from the electrode pad controller 103D (S105). It should be noted that after the electrocardiogram analysis is started, the electrode pad controller 103D determines whether the electrode pads 14 and 15 have been attached to the injured or sick person or not. In a case where it is determined that the electrode pads 14 and 15 have been detached, the electrode pad controller 103D performs a guidance to the rescuer on how to attach the electrode pads 14 and 15 to the injured or sick person in a similar manner to that in the step S103. When the electrode pads 14 and 15 are attached again, the electrocardiogram analysis part 106 performs the electrocardiogram analysis again.

Next, the electrode pad controller 103D determines whether an electric shock is necessary or not, based on a result of the electrocardiogram analysis of the electrocardiogram analysis part 106 (S106). When the electrode pad controller 103D determines that the electric shock is necessary, the power supply controller 103C determines whether the charging of the internal capacitor of the high voltage unit 105 has been completed or not (S107). When the power supply controller 103C determines that the charging of the internal capacitor has been completed, the sound controller 103B starts output of a warning sound indicating a state in which the AED body 10 has been internally charged with high voltage, to the rescuer (S108). The warning sound is, for example, a continuous buzzer sound. By hearing the warning sound, the rescuer can recognize that there is a possibility that the high voltage will be discharged from the AED body 10.

In addition to the step S108, the display controller 103A and the sound controller 103B start a pre-discharge alert guidance to the rescuer (S109). Details of the alert guidance before discharging will be described later. It should be noted that the display controller 103A and the sound controller 103B may start the step S109 prior to the step S108.

When it is determined that the electrode pads 14 and 15 have been attached to the injured or sick person, the power supply controller 103C performs discharging on the injured or sick person (S110). The power supply controller 103C may automatically perform the discharging after the pre-discharging alert guidance has been performed, or may be configured to perform the discharging as soon as a discharge button (unillustrated) has been depressed by the rescuer.

After having performed the discharging, the controller 103 stops the pre-discharging alert guidance and the output of the warning sound (S111). The controller 103 repeats the aforementioned steps S102 to S111 (S101). When the electrode pad controller 103D determines, in the step S106, that the discharging is unnecessary based on the result of the electrocardiogram analysis of the electrocardiogram analysis part 106, the controller 103 terminates the rescue process (S112).

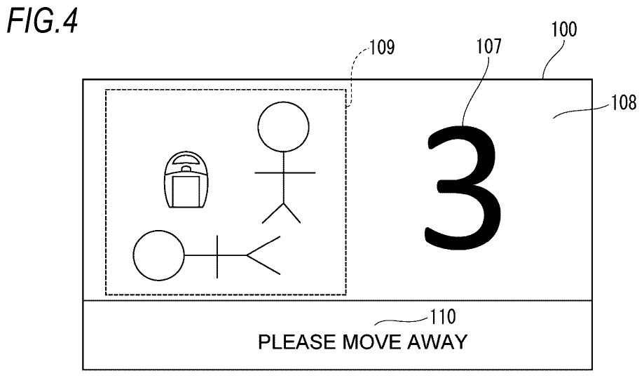

An example of the pre-discharging alert guidance (S109) of FIG. 3 will be described with reference to FIG. 4 that illustrates the display 100. The display 100 includes a timing displaying section 107, a background section 108, an illustration displaying section 109, and a warning text displaying section 110. The timing display 107, the background display 108, the illustration display 109, and the warning text display 110 perform the following process as an example of the pre-discharging alert guidance (S109) illustrated in FIG. 3.

The timing display 107 presents time information regarding a time length until the discharging is performed. The time information is a count character suitably indicating, as a numerical value, a remaining time until the electric discharge. The time information will be described as follows using the count character. It should be noted that the time information (count character) may be information with one or more decimal places. The background display 108 presents a background color, a background pattern, etc. Furthermore, the background display 108 changes the background color, the background pattern, etc. so as to inform the rescuer of the danger of receiving an electric shock due to the execution of the discharging. The illustration display 109 presents, for example, an illustration illustrated in FIG. 4, so as to prompt the rescuer to move away from the injured or sick person. The warning text display 110 presents, for example, a text such as “Please move away” illustrated in FIG. 4, so as to prompt the rescuer to move away from the injured or sick person. Here, the expression “illustration” means an illustration of human beings such as the rescuer and the injured or sick person and a device such as the AED. A background area other than the human beings and the device inside a dashed line designated by a reference sign 109 illustrated in FIG. 4 is not included in the illustration display 109, but should be included in the background display 108.

As an example of the aforementioned alert guidance (S109), the display controller 103A illustrated in FIG. 2 changes a color presented on the display 100 in accordance with the time length until the discharging is performed. Here, the expression “color” means at least one of hue, chroma, and brightness. It should be noted that a pattern (plain or hatched) presented on the display 100 may be changed instead of changing the color presented on the display 100. According to the present configuration, the rescuer can easily notice the change in the display screen of the display 100, and recognize that the electric discharge will be started.

The display controller 103A may change the color presented on the display 100 at a predetermined time interval (e.g. an interval of 0.5 seconds) shorter than a time interval at which the numerical value of the time information (count character) changes. When, for example, the color presented on the display 100 changes at the interval of 0.5 seconds, a color presented on the display 100 changes to another color during displaying the same time information (count character). It should be noted that the time information mentioned herein is information including a discrete numerical value. According to the present configuration, the color of the time information (count character), etc. changes even while the numerical value of the time information (count character) presented on the display 100 is not updated. Accordingly, the rescuer can easily notice the change in the display screen of the display 100, and recognize that the electric discharge will be started.

In addition, the display controller 103A may change the color presented on the display 100 at a timing when the numerical value of the time information until the discharging is performed changes. It should be noted that the time information mentioned herein is information including a discrete numerical value.

According to the present configuration, at the timing when the numerical value of the time information (count character) until the discharging is performed changes, the rescuer can easily notice the change in the display screen of the display 100, and recognize that the discharging will be started.

In addition, in accordance with the time length until the discharging is performed, the display controller 103A may partially or entirely change at least one of the color and the pattern regarding at least one of the time information (count character) presented on the timing display 107, the background color, the background pattern, etc. presented on the background display 108, the illustration information presented on the illustration display 109, and the text information presented on the warning text display 110. According to the present configuration, at least one of the color and the pattern of the time information (count character) presented on the timing display 107, etc. is changed so that the rescuer who is watching the display 100 can be facilitated to understand the current time information (count character) and a fact that the electric discharge will be started. According to the present configuration, the background color, etc. of the background display 108 may be alternatively changed so that the rescuer who is not watching the display 100 can be facilitated to realize the change in the display 100 and understand a fact that the electric discharge will be started.

In addition, the display controller 103A may partially or wholly change the color regarding at least one of the time information (count character) presented on the timing display 107, the background color, the background pattern, etc. presented on the background display 108, the illustration information presented on the illustration display 109, and the text information presented on the warning text display 110 so that an angle of a hue difference between the color and the changed color is more than 45° in a hue circle divided into 24 equal parts. According to the present configuration, the hue of the time information (count character) etc. is changed to a hue that is not a similar hue, so that the change in the display 100 can be made so conspicuous that the rescuer can easily recognize the change in the display 100 and understand a fact that the electric discharge will be started.

Another example of the pre-discharging alert guidance (S109) illustrated in FIG. 3 will be described with reference to FIG. 2 again. The sound generator 101 illustrated in FIG. 2 outputs a warning sound indicating the state in which the internal capacitor of the high voltage unit 105 has been charged with high voltage, and a timing sound until the discharging is performed. As used herein, the term “timing sound” means, for example, a count-down sound including the discrete numerical value such as “3”, “2”, or “1”.

The sound controller 103B changes the warning sound in accordance with presence or absence of output of the timing sound. That is, the sound controller 103B may change at least one of volume, scale, and tone of the warning sound in accordance with the presence or absence of output of the timing sound. According to the present configuration, even while the warning sound is outputted, the rescuer can still easily notice the timing sound and easily understand a fact that the electric discharge will be started.

Specifically, the sound controller 103B may set the volume of the warning sound during the output of the timing sound to a level lower than the volume of the warning sound during the non-output of the timing sound or to zero, or may set the scale of the warning sound during the output of the timing sound to a level lower than the scale of the warning sound during the non-output of the timing sound, or may make the tone of the warning sound during the output of the timing sound different from the tone of the warning sound during the non-output of the timing sound. According to the present configuration, the volume of the warning sound is reduced during the output of the timing sound. In this manner, even while the warning sound is outputted, the rescuer can easily recognize the timing sound and understand a fact that the electric discharge will be started. Alternatively, the scale of the warning sound may be lowered during the output of the timing sound. In this manner, even while the warning sound is outputted, the rescuer can easily recognize the timing sound and understand a fact that the electric discharge will be started. Alternatively, the tone of the warning sound during the output of the timing sound may be made different from the tone of the warning sound during the non-output of the timing sound. In this manner, even while the warning sound is outputted, the rescuer can easily recognize the timing sound and understand a fact that the electric discharge will be started.

In addition, the sound controller 103B may change at least one of the volume, the scale and the tone of the timing sound in accordance with the number of times of the output of the timing sound. It should be noted that regarding the number of times of the output of the timing sound, for example, assume that the timing sound is a count-down sound, and the numbers “3”, “2”, and “1” are outputted with sound. In this case, the number of times of the output of the timing sound is three. According to the present configuration, as the number of times of the output of the timing sound increases, the volume of the timing sound is increased, the scale of the timing sound is raised, or the tone of the timing sound is made different from the tone of the warning sound. In this manner, even while the warning sound is outputted, the rescuer can easily recognize the timing sound and understand a fact that the electric discharge will be started.

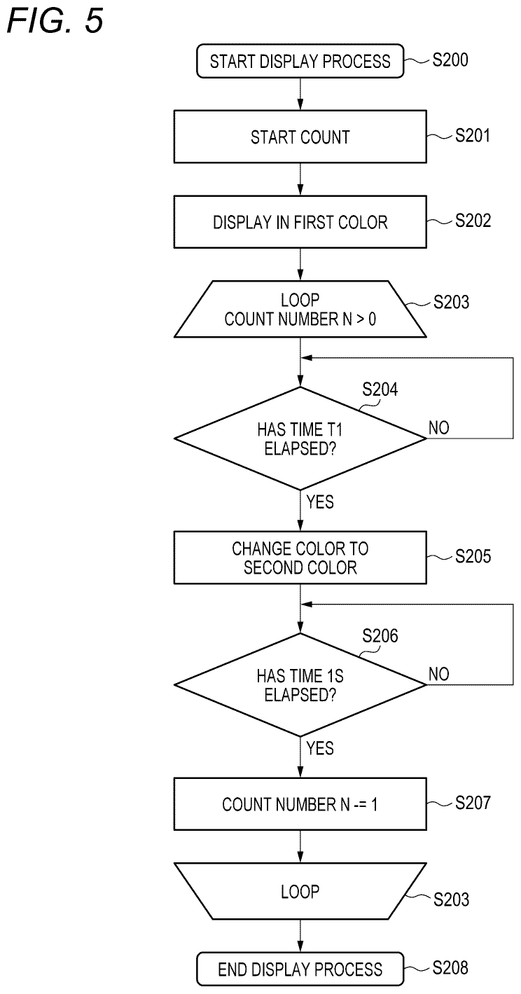

FIG. 5 illustrates a flow of a display process of the AED according to a first embodiment of the present disclosure. As an example of the alert guidance (S109) illustrated in FIG. 3, the display controller 103A illustrated in FIG. 2 performs the display process of changing a color presented on the display 100 in accordance with a time length until discharging is performed (S200).

The controller 103 starts count of a time length until the discharging is performed (S201). The timing display 107 presents time information (count character) in a first color in accordance with the start of the count (S202). It should be noted that, instead of the timing display 107 presenting the time information (count character) in the first color, the background display 108 may present a background color, a background pattern, etc. in the first color, the illustration display 109 may present the aforementioned illustration information in the first color, or the warning text display 110 may present the aforementioned warning text information in the first color.

Next, after a predetermined time length between zero seconds and one second has elapsed since the start of the count (S204), the timing display 107 changes the color of the time information (count character) from the first color to a second color (S205). Next, after one second has elapsed since the start of the count (S206), the controller 103 updates a numerical value of the time information (count character) (S207). Specifically, the controller 103 subtracts one from the numerical value N of the time information (count character). When the numerical value N of the updated time information (count character) is larger than zero, the controller 103 repeats the steps S204 to S207. When the numerical value N of the updated time information (count character) is zero, the display controller 103A terminates the display process (S208). It should be noted that the numerical value N of the time information (count character) is a discrete numerical value.

In the present embodiment, the timing display 107 is configured to change the color of the time information (count character) from the first color to the second color (S205). However, the timing display 107 may be alternatively configured to change the color of the time information (count character) several times, i.e. further change the color of the time information (count character) from the second color to a third color during the update of the numerical value of the time information (count character). It should be noted that instead of changing the color, the background or a pattern (plain or hatched) of the time information (count character) may be changed.

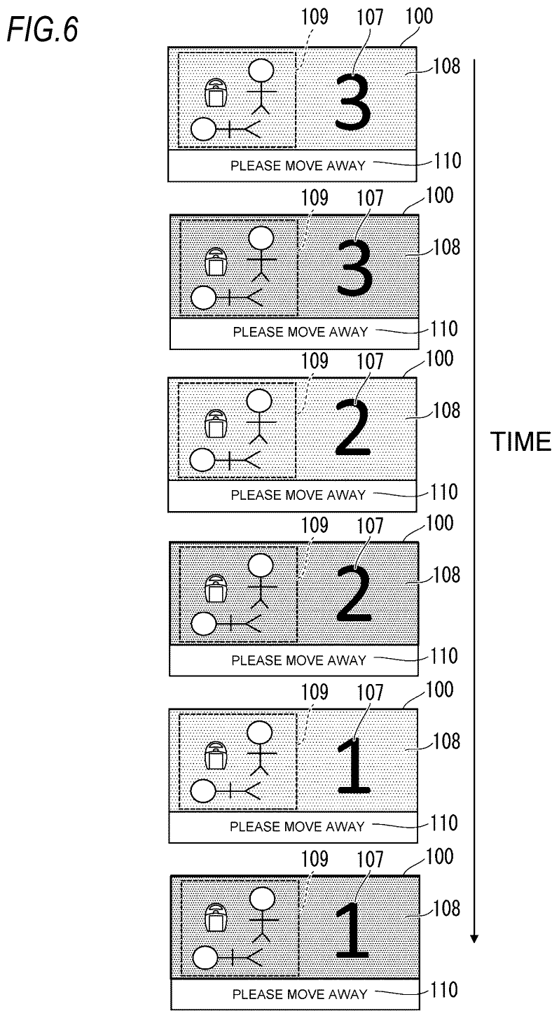

FIG. 6 illustrates a transition flow of the display of the AED according to the first embodiment of the present disclosure. In FIG. 6, the vertical axis represents an elapse of time, and a flow of a change in the background color presented on the background display 108 is shown with the passage of the time until the discharging is performed. The display controller 103A illustrated in FIG. 2 changes the background color presented on the background display 108 alternately between the first color and the second color at a predetermined time interval (e.g. an interval of 0.5 seconds) shorter than a time interval (e.g. an interval of one second) at which the numerical value of the time information (count character) changes. In this case, two background colors, i.e. the first color and the second color, are presented on the background display 108 during the period of time that one and the same count character is being displayed.

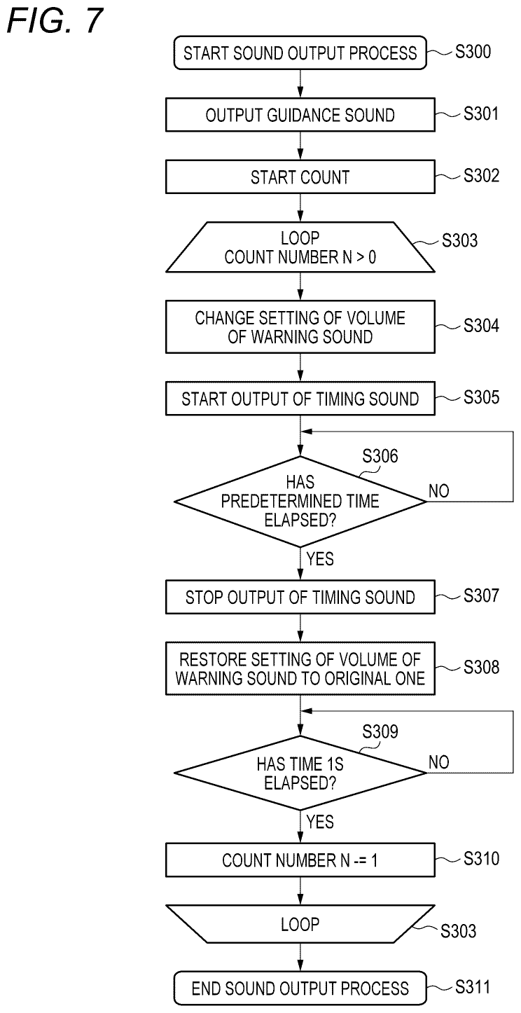

FIG. 7 illustrates a flow of a sound output process of the AED according to a second embodiment of the present disclosure. As another example of the alert guidance (S109) illustrated in FIG. 3, the sound controller 103B performs the sound output process of changing a warning sound in accordance with presence or absence of output of a timing sound (S300).

The sound generator 101 outputs a guidance sound such as “Please move away” so as to prompt the rescuer to move away from the injured or sick person who will be subjected to discharging (S301).

Next, the controller 103 starts a count-down until the discharging is performed (S302). After the start of the count-down, the sound controller 103B changes setting of volume of the warning sound (S304). In addition to the change in the volume of the warning sound to be outputted, the sound generator 101 starts output of the timing sound (S305). Here, the timing sound may be a sound indicative of, with a numerical value etc., a time length until the discharging is performed, or may be a beep sound. The sound generator 101 reduces the volume of the warning sound, and then outputs the timing sound. In this manner, the rescuer can easily hear both the timing sound and the warning sound and understand a fact that the electric discharge will be started.

Next, after a lapse of a predetermined time length since the start of the count (S306), the sound generator 101 stops the output of the timing sound (S307), and the sound controller 103B restores the setting of the volume of the warning sound to original one (S308). Thus, the sound generator 101 outputs only the warning sound at the original volume setting. Next, after a lapse of one second since the start of the count (S309), the controller 103 updates a numerical value of time information (count character) (S310). Specifically, the controller 103 subtracts one from the numerical value N of the time information (count character). When the numerical value N of the updated time information (count character) is larger than zero, the controller 103 repeats the steps S304 to S310. When the numerical value N of the updated time information (count character) is zero, the sound controller 103B terminates the sound output process (S311).

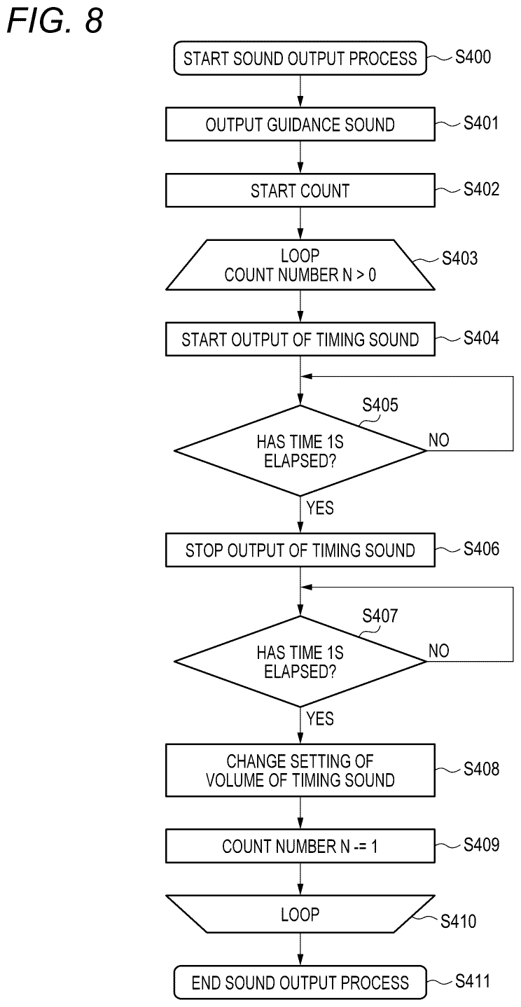

FIG. 8 illustrates a flow of a sound output process of the AED according to a third embodiment of the present disclosure. As another example of the alert guidance (S109) illustrated in FIG. 3, the sound controller 103B performs the sound output process of changing a warning sound in accordance with the number of times of output of a timing sound (S400).

The sound generator 101 outputs a guidance sound such as “Please move away” to prompt the rescuer to move away from the injured or sick person who will be subjected to discharging (S401).

Next, the controller 103 starts a count-down until the discharging is performed (S402). After the start of the count-down, the sound generator 101 starts output of the timing sound while changing the volume of the warning sound to be outputted (S404). Here, the timing sound may be a sound indicative of, with a numerical value etc., a time length until the discharging is performed, or may be a beep sound.

Next, after a lapse of a predetermined time length between zero seconds and one second since the start of the count (S405), the sound generator 101 stops the output of the timing sound (S406). After a lapse of one second since the start of the count (S407), the sound controller 103B changes setting of volume of the timing sound (S408), and the controller 103 updates a numerical value of time information (count character) (S409). When the numerical value N of the updated time information (count character) is larger than zero, the controller 103 repeats the steps S404 to S409. In this manner, the sound controller 103B changes the setting of the volume of the timing sound every time when the numerical value of the time information (count character) is updated, i.e., every time when the number of times of the output of the timing sound increases. As the number of times of the output of the timing sound increases, the sound controller 103B increases the volume of the timing sound outputted by the sound generator 101. In this manner, even while the warning sound is outputted, the rescuer can easily recognize a count sound and understand a fact that the start of the electric discharge is imminent.

The embodiment of the present disclosure has been described above. Needless to say, the technical scope of the present disclosure should not be interpreted as limited by the description of the present embodiment. The present embodiment is merely exemplified, and it is understood by those skilled in the art that various modifications can be made on the embodiment within the scope of the invention described in CLAIMS. The technical scope of the present disclosure should be defined based on the scope of the invention described in the CLAIMS and its equivalent scopes.

The present application is based on a Japanese patent application No. 2022-001892 filed on Jan. 7, 2022, the entire contents of which are hereby incorporated by reference.

1. An automated external defibrillator configured to deliver an electric shock for defibrillation to a heart of a subject, the automated external defibrillator comprising:
a sound generator configured to output;
a timing sound expressing, by sound, a timing until the electric shock is delivered; and
a warning sound expressing a state in which an internal capacitor of the automated external defibrillator has been charged with high voltage; and
a sound controller configured to control the sound generator,
wherein the sound controller changes the warning sound in accordance with presence or absence of output of the timing sound.

2. The automated external defibrillator according to claim 1,
wherein the sound controller changes at least one of volume, scale, and tone of the warning sound in accordance with presence or absence of output of the timing sound.

3. The automated external defibrillator according to claim 2,
wherein the sound controller sets the volume of the warning sound during output of the timing sound to a level lower than the volume of the warning sound during non-output of the timing sound.

4. The automated external defibrillator according to claim 2,
wherein the sound controller sets the scale of the warning sound during output of the timing sound to a level lower than the scale of the warning sound during non-output of the timing sound.

5. The automated external defibrillator according to claim 2,
wherein the sound controller makes the tone of the warning sound during output of the timing sound different from the tone of the warning sound during non-output of the timing sound.

6. The automated external defibrillator according to claim 1,
wherein the sound controller changes at least one of volume, scale, and tone of the timing sound in accordance with a number of times of output of the timing sound.

7. The automated external defibrillator according to claim 6,
wherein the sound controller increases the volume of the timing sound in accordance with the number of times of output of the timing sound.

8. The automated external defibrillator according to claim 6,
wherein the sound controller raises the scale of the timing sound in accordance with the number of times of output of the timing sound.

9. The automated external defibrillator according to claim 1,
wherein at least before output of the timing sound, the sound controller outputs a guidance sound from the sound generator; and
wherein the guidance sound calls attention to a rescuer so as to prompt the rescuer to move away from the subject.

10. The automated external defibrillator according to claim 1, further comprising:
a display configured to present:
at least one of time information regarding a time length until the electric shock is delivered;
an illustration regarding reception of the electric shock; and
a warning text regarding the reception of the electric shock; and
a display controller configured to control the display,
wherein the display controller changes a color presented on the display in accordance with the time length until the electric shock is delivered.
